(19) United States
(12) Patent Application Publication
(10) Pub. No.: US 2025/0387231 A1
(43) Pub. Date: Dec. 25, 2025

(54) A SURGICAL IMPLANT

(71) Applicant: OSTEOPORE INTERNATIONAL PTE. LTD., Singapore (SG)

(72) Inventors: Pei Hao Frederick CHING, Singapore (SG); Jiong Le LAM, Singapore (SG); Marie-Luise WILLE, Jindalee (AU); Buddhi Yushendra HERATH, South Brisbane (AU); Sinduja SURESH, South Brisbane (AU); Jing LIM, Singapore (SG); Dietmar Werner HUTMACHER, Bellbowrie (AU)

(21) Appl. No.: 18/881,307

(22) PCT Filed: Jul. 5, 2023
(86) PCT No.: PCT/SG2023/050471

§ 371 Date: Jan. 6, 2025

(30) Foreign Application Priority Data
Jul. 6, 2022 (SG) 10202250375Y

(51) Int. Cl. A61F 2/28; A61F 2/30; A61L 27/18

(52) U.S. Cl. CPC A61F 2/28; A61F 2/30942; A61L 27/18; A61F 2002/2825; A61F 2002/2835; A61F 2002/2853; A61F 2002/2892; A61F 2002/30062; A61F 2002/30153; A61F 2002/30224; A61F 2002/30331; A61F 2002/30599; A61F 2002/30616; A61F 2002/30948; A61F 2002/30985; A61L 2430/02

(57) ABSTRACT

The present disclosure relates to surgical implants. In particular, the disclosure relates to a surgical implant modular assembly comprising at least a first scaffold and a second scaffold.

### CROSS-REFERENCE TO RELATED APPLICATIONS

The present application claims the benefit of Singapore Patent Application No. 10202250375Y, filed on 6 Jul. 2022, the disclosure of which is hereby incorporated in its entirety by reference herein.

### FIELD OF THE DISCLOSURE

The present disclosure generally relates to surgical implants. In particular, the disclosure relates to a surgical implant modular assembly comprising at least a first scaffold and a second scaffold.

### BACKGROUND

Bone voids or defects may be created from a traumatic event, surgical resection of cancerous or infected tissue. However, predetermination of bone voids size is challenging due to the severity of the injury and amount of bone tissues to be removed.

Efforts have been focused to develop and manufacture a patient specific or customized implant for patients to provide a treatment solution for bone defects that matches the patient's anatomy to achieve optimal healing due to the better fitting and conformity to the unique geometry of bone voids. The currently available solution, however, comes with long turnaround time from its designing, developing, testing and fabrication.

In view of the above, there is a need to develop surgical implants and related methods that overcome or at least ameliorate the limitations described above.

### SUMMARY

In one aspect there is provided a surgical implant modular assembly. According to some embodiments, the surgical implant modular assembly may include a first scaffold and a second scaffold. The first scaffold may be structurally identical to the second scaffold. According to some embodiments, each of the first and the second scaffold may have a first section and a second section. For each of the first and the second scaffold, the first section may be integrated to the second section. Further, the first section of each of the first scaffold and second scaffold may be positioned above the second section of the same scaffold. Additionally, the second section of the first scaffold may have a first opening. The second section of the second scaffold may have a second opening. The second opening may be configured to complement the first section of the first scaffold such that when the second scaffold is stacked above the first scaffold, the first section of the first scaffold fits into the second opening thereby forming said surgical implant modular assembly.

Optionally, the modular assembly may be a cylindrical modular assembly. Each of the first opening and the second opening may be a sideway opening in a range from 60° to 120°. Optionally, the first scaffold may further comprise a first pin disposed on top of the first section of the first scaffold and a first slot disposed on bottom of the second section of the first scaffold. Still optionally, the second scaffold may further comprise a second pin disposed on top of the first section of the second scaffold and a second slot disposed on bottom of the second section of the second scaffold.

According to some embodiments of the disclosure, the surgical implant modular assembly may further comprise an alternate scaffold having a third slot such that when the alternate scaffold is stacked above the second section of the second scaffold, the first pin of the first scaffold may be fitted into the third slot of the alternate scaffold. Optionally, the surgical implant modular assembly described herein may further comprise a second alternate scaffold having a third pin such that when the second alternate scaffold is stacked below the second scaffold, the third pin of the second alternate scaffold may be fitted into the second slot of the second scaffold. According to some embodiments of the disclosure, the first pin, the second pin, the third pin, the first slot, the second slot and the third slot, each may be of rectangular shape. The surgical implant modular assembly may optionally comprise a bioresorbable material. Said bioresorbable material may be a porous bioresorbable material having a pore size of 0.4 mm to 4 mm. Said bioresorbable material may be a polymer, a salt or a composite. Optionally, the polymer or the composite may comprise a polycaprolactone (PCL)-based polymer. Optionally, the composite may be doped with one or more metals.

According to some embodiments of the disclosure, the surgical implant modular assembly described herein may be useful for treating a bone defect in a subject. The bone defect may be a long bone defect selected from the group consisting of femur, tibia and humerus. Optionally, size of each of the first scaffold, the second scaffold, the alternate scaffold and the second alternate scaffold of the surgical implant modular assembly may be customized to individual subject. The size of each of the first scaffold, the second scaffold, the alternate scaffold and the second alternate scaffold may be determined from a Computed tomography (CT) scan. Optionally, the customized scaffold may be prepared via an additive manufacturing.

In another aspect, there is provided a method for treating a bone defect in a subject, comprising inserting the surgical implant modular assembly described in the present disclosure to the subject. The bone defect may be a long bone defect selected from the group consisting of femur, tibia and humerus.

In another aspect, there is provided a method for manufacturing the surgical implant modular assembly described in the present disclosure. The method may comprise determining a suitable size of the surgical implant. The method may further include mixing one or more reagents to form each of the first and second scaffold followed by stacking the second scaffold above the first scaffold, thereby forming the surgical implant modular assembly.

### BRIEF DESCRIPTION OF THE DRAWINGS

The present disclosure will be understood and better appreciated from the following detailed description taken in conjunction with the drawings. Identical structures, elements or parts, which appear in more than one figure, are generally labeled with the same or similar number in all the figures in which they appear, wherein:

FIG. 1 is a perspective view of the first or second scaffold of the surgical implant modular assembly, according to some embodiments of the disclosure;

FIG. 2A is a top view of the first or second scaffold of the surgical implant modular assembly, according to some embodiments of the disclosure;

FIG. 2B is a front view of the first or second scaffold of the surgical implant modular assembly, according to some embodiments of the disclosure;

FIG. 2C is a side view of the first or second scaffold of the surgical implant modular assembly, according to some embodiments of the disclosure;

FIG. 3A is a perspective view of the surgical implant modular assembly that has been assembled, according to a first embodiment of the disclosure;

FIG. 3B is a perspective view of the exploded surgical implant modular assembly showing the first scaffold, second scaffold, first alternate scaffold and second alternate scaffold, according to the first embodiment of the disclosure;

FIG. 4A is an image illustrating a perspective view of the assembled surgical implant modular assembly described in FIG. 3A;

FIG. 4B is an image illustrating a top view of the individual parts of the surgical implant modular assembly described in FIG. 3A to be assembled including the first scaffold, second scaffold, first alternate scaffold and second alternate scaffold;

FIG. 4C is an image illustrating a perspective view of FIG. 4B;

FIG. 5A is a perspective view of the surgical implant modular assembly that has been assembled, according to the second embodiment of the disclosure;

FIG. 5B is a perspective view of the exploded surgical implant modular assembly showing the first scaffold, second scaffold, first alternate scaffold and second alternate scaffold, according to the second embodiment of the disclosure;

FIG. 6A is an image illustrating a perspective view of the assembled surgical implant modular assembly described in FIG. 5A;

FIG. 6B is an image illustrating a top view of the individual parts of the surgical implant modular assembly described in FIG. 5A to be assembled including the first scaffold, second scaffold, first alternate scaffold and second alternate scaffold; and

FIG. 6C is an image illustrating a perspective view of FIG. 6B.

### DETAILED DESCRIPTION

The present disclosure provides a surgical implant modular assembly that may be used as bone void filler. Such a bone void filler may be structured while allowing users, for example surgeons, to assemble the surgical implant modular assembly into its desired length and size to fill the bone void. The customization of the surgical implant may be possible as it is being created during surgery. This is beneficial as the customization may allow the production of individually manufactured fillers of various lengths and sizes.

In some embodiments of the disclosure, the first scaffold and the second scaffold may be structurally identical or similar. The degree of similarity between the first scaffold and the second scaffold may be more than 80% such as 82%, 85%, 86%, 88%, 90%, 92%, 95%, 96%, 97% 98%, 99% or 100%. In some embodiments, the degree of similarity between the first scaffold and the second scaffold is preferably 100%. The first scaffold may have a first section and a second section. In some embodiments, the first section and the second section are integrated or joined. In some embodiments, the first section is positioned above the second section. The first section of the first scaffold may be distinct from the second section of the first scaffold. Similarly, the first section of the second scaffold may be distinct from the second section of the second scaffold. When the first scaffold is structurally identical to the second scaffold, the second scaffold also has the first section and the second section, wherein the first section and the second section are integrated or joined. In some embodiments of the second scaffold, the first section is positioned above the second section. In some embodiments, the first section of the first scaffold may be identical to the first section of the second scaffold. Likewise, the second section of the first scaffold may be identical to the second section of the second scaffold.

In some embodiments, the surgical implant modular assembly may be of cylindrical shape. Other suitable shapes may be used provided it has the same benefits as the cylindrical surgical implant modular assembly. In some embodiments, when the surgical implant modular assembly has a cylindrical shape, the surgical implant modular assembly may further comprise a cavity. Such cavity may be provided in the form of an inner tube. Such inner tube may use an intramedullary nail as an external fixation device.

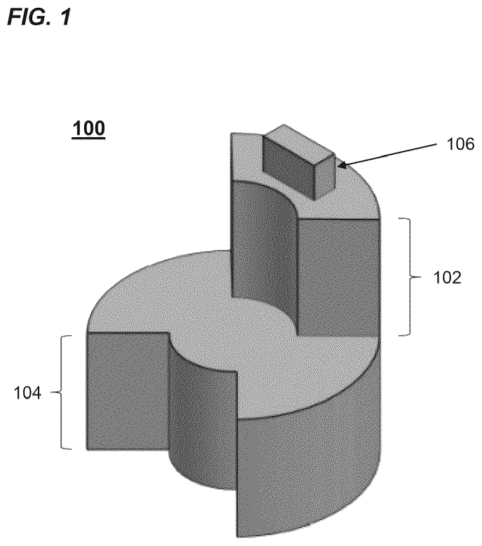

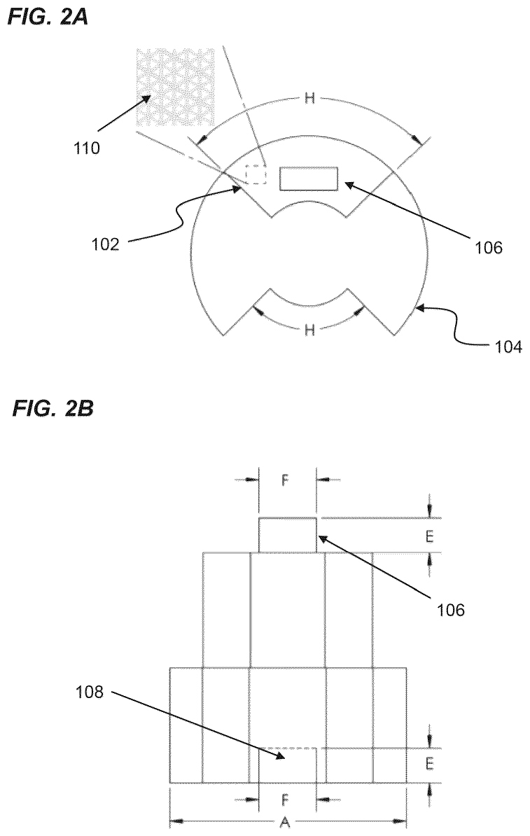

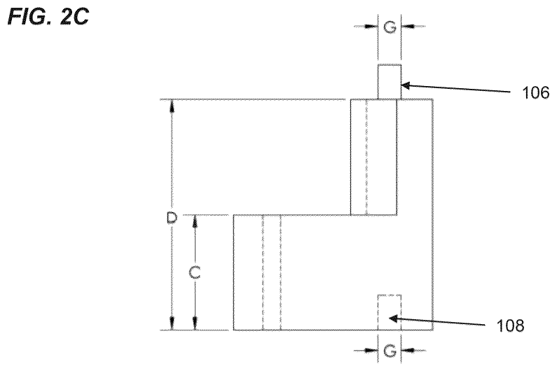

In some embodiments, the second section of the first scaffold has an opening referred as a first opening. Similarly, the second section of the second scaffold has an opening referred as a second opening. When the first scaffold is identical to the second scaffold, it is to be understood that the first opening is likewise identical to the second opening. For the cylindrical surgical implant modular assembly, the first opening or the second opening may be a sideway opening. For clarity, in the case of the cylindrical surgical implant modular assembly, the top view of such surgical implant will have a cut out portion corresponding to the opening such as the one shown in FIG. 1 or FIG. 2A. Without being bound by theory, the sideway opening for the cylindrical surgical implant modular assembly may refer to a cut out at radial periphery of the cylinder. In some embodiments, the sideway opening of the first or second opening may form an angle in a range from about 60° to about 120° for example 60°, 65°, 70°, 75°, 80°, 85°, 90°, 95°, 100°, 105°, 110°, 115°, and 120° measured from the center radius of the cylinder. Any other angles not stated but within the range stated may also be used. The range of the angle above may advantageously allow the insertion of the surgical implant into the bone defects with or without prior implantation of intramedullary nail or other suitable internal fixation device.

In some embodiments, the first scaffold may be stacked above the second scaffold. In some embodiments, the second scaffold may be stacked above the first scaffold. When the first scaffold is stacked above the second scaffold, the first opening may be configured to complement the first section of the second scaffold such that the first section of the second scaffold may fit into the first opening thereby forming said surgical implant modular assembly. In some embodiments, when the second scaffold is stacked above the first scaffold, the second opening may be configured to complement the first section of the first scaffold such that the first section of the first scaffold may fit into the second opening thereby forming said surgical implant modular assembly.

In some embodiments of the surgical implant modular assembly, the first scaffold further comprises a first pin disposed on top of the first section of the first scaffold and a first slot disposed on bottom of the second section of the first scaffold. In some embodiments, each of the first pin and first slot is of rectangular shape.

In some embodiments of the surgical implant modular assembly, the second scaffold further comprises a second pin disposed on top of the first section of the second scaffold and a second slot disposed on bottom of the second section of the second scaffold. In some embodiments, each of the second pin and second slot is of rectangular shape.

In some embodiments, the surgical implant modular assembly may further comprise an alternate scaffold having a third slot such that when the second scaffold is stacked above the first scaffold and the alternate scaffold is stacked above the second section of the second scaffold, the first pin of the first scaffold may be fitted into the third slot of the alternate scaffold. In some embodiments, the third slot is of rectangular shape.

In some embodiments, the surgical implant modular assembly may further comprise a second alternate scaffold having a third pin such that when the second scaffold is stacked above the first scaffold and the second alternate scaffold is stacked below the first scaffold, the third pin of the second alternate scaffold may be fitted into the first slot of the first scaffold. In some embodiments, the third pin is of rectangular shape. It is to be understood that the first pin, the second pin, the third pin, the first slot, the second slot and the third slot, each may be of other shapes than rectangular including cylindrical, triangle, pentagonal and hexagonal.

In an exemplary embodiment of the disclosure, as can be seen from FIG. 1, the first or second scaffold of the surgical implant modular assembly disclosed herein is denoted as 100. In this embodiment, first section 102 of scaffold 100 is positioned above second section 104 of scaffold 100. For clarity, first section 102 of scaffold 100 shown in FIG. 1 is positioned above second section 104 of scaffold 100 in vertical direction. First section 102 and second section 104 of scaffold 100 are integrated or joined. It can be also seen from FIG. 1, pin 106 of scaffold 100 as well as sideway opening of the first section and that of the second section. The sideway opening is characterized by the angled formed as described herein. As can be seen from FIG. 2A, depicting top view of the first or second scaffold 100, the angle characterizing the sideway opening of second section 104 is H. In such embodiment, the angle characterizing the sideway opening of first section 102 is (360-H) so that when the second scaffold is stacked above the first scaffold, the first section of the first scaffold will fit into the second opening of the second scaffold. For the surgical implant modular assembly consisting of the first or second scaffold shown in FIG. 1, the surgical implant modular assembly is substantially cylindrical. Top view of the first or second scaffold 100 depicts that the scaffold consists of a network of bioresorbable material 110 as will be described below.

FIG. 2B describes the front view of the first or second scaffold of the surgical implant modular assembly disclosed herein. In the same drawing, A describes the diameter of the second section. Pin 106 of scaffold 100 is characterized by its height (E) and width (F). Pin 106 is disposed on top of the first section 104 of scaffold 100. Further, pin 106 may be further characterized by its thickness (G) as can be seen from FIG. 2C describing the side view of the first or second scaffold of the surgical implant modular assembly disclosed herein. It is to be appreciated by a person skilled in the art that each of E, F and G can be varied or adjusted as necessary. FIG. 2B further shows slot 108 disposed on bottom of the second section 104 of the scaffold. To facilitate the assembly of the first scaffold and the second scaffold, the dimension of the slot is shaped to match the dimension of the pin. In FIG. 2B, both pin and slot have width of F and height of E and as can be seen in FIG. 2C, both have thickness of G.

FIG. 2C describes the side view of the first or second scaffold of the surgical implant modular assembly disclosed herein. In this drawing, it can be seen that first section 102 and second section 104 are characterized by having a total height of D. In some embodiments, the height of second section 104 is C. Hence, the height of the first section 102 is (D-C). It is to be appreciated by a person skilled in the art that each of C and D can be varied or adjusted as necessary.

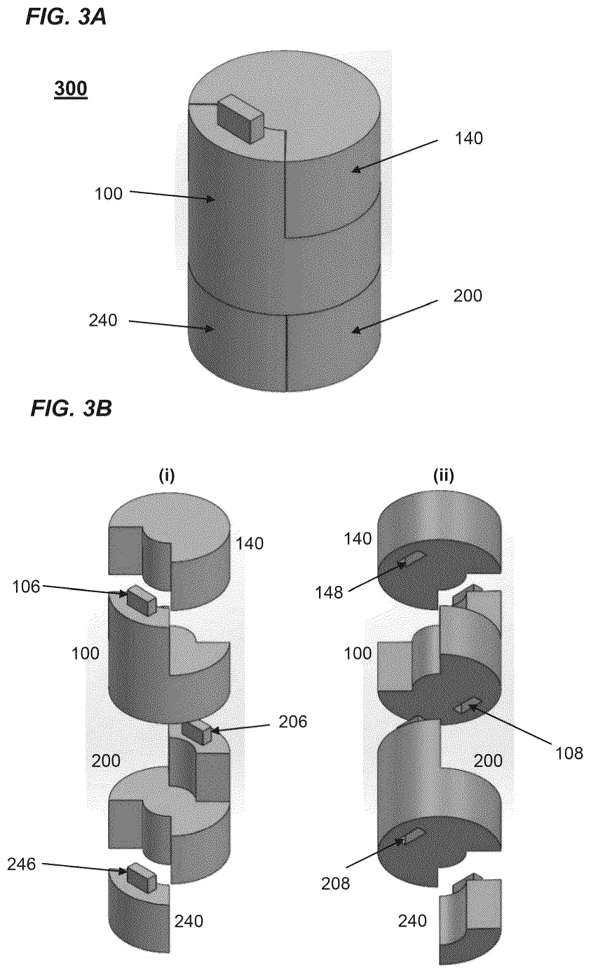

FIG. 3A describes a first embodiment of the surgical implant modular assembly 300 disclosed herein. In this drawing, parts of the surgical implant modular assembly have been already assembled and ready for use. Thus, the surgical implant modular assembly 300 consists of first scaffold 200, second scaffold 100, first alternate scaffold 140 and second alternate scaffold 240. The opening of second scaffold 100 is configured to complement first section of first scaffold 200 such that when second scaffold 100 is stacked above first scaffold 200, first section of first scaffold 200 fits into second opening. The first alternate scaffold 140 is stacked above the second section of the second scaffold 100 such that first pin (206 in FIG. 3B(i)) of first scaffold 200 is fitted into the slot (148 in FIG. 3B(ii)) of the first alternate scaffold 140. The second alternate scaffold 240 has a pin (246 in FIG. 3B(i)) such that when second alternate scaffold 240 is stacked below second scaffold 100, pin 246 (FIG. 3B(i)) of second alternate scaffold 240 is fitted into slot 108 (FIG. 3B(ii)) of second scaffold 100.

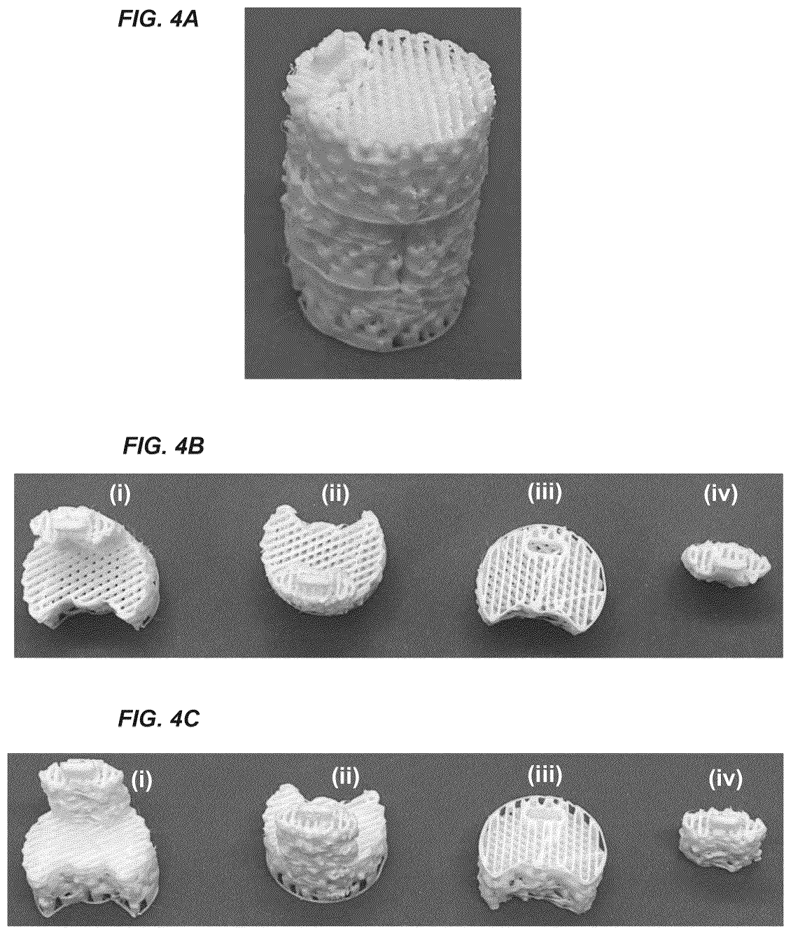

FIGS. 4A-C depict the surgical implant modular assembly described in FIGS. 3A-B. FIG. 4A depicts the surgical implant modular assembly with its parts thereof assembled. FIGS. 4B-C show the top view and perspective view of the individual parts of the surgical implant modular assembly described in FIG. 4A, respectively.

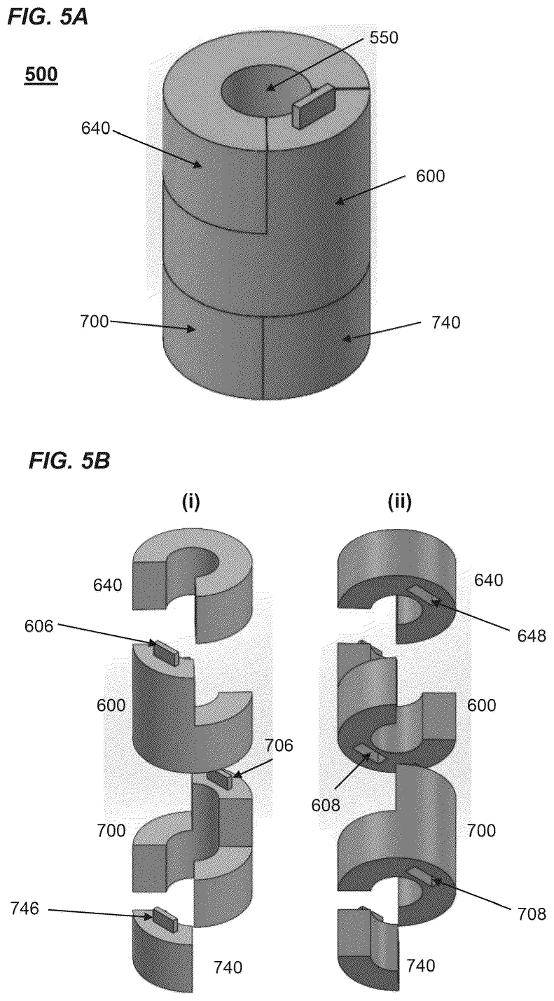

FIG. 5A describes a second embodiment of the surgical implant modular assembly 500 disclosed herein. As can be seen, the surgical implant modular assembly of the second embodiment is similar to that of the first embodiment, except that in the second embodiment, the modular assembly further consists of a cavity 550. Such cavity is provided in the form of an inner tube 550. In this drawing, parts of the surgical implant modular assembly have been already assembled and ready for use. Thus, the surgical implant modular assembly 500 consists of first scaffold 700, second scaffold 600, first alternate scaffold 640 and second alternate scaffold 740. The first alternate scaffold 640 is stacked above the second section of the second scaffold 600 such that the first pin (706 in FIG. 5B(i)) of the first scaffold 700 is fitted into the slot (648 in FIG. 5B(ii)) of the first alternate scaffold 640. The second alternate scaffold 740 has a pin (746 in FIG. 5B(i)) such that when the second alternate scaffold 740 is stacked below second scaffold 600, pin 746 (FIG. 5B(i)) of the second alternate scaffold 740 is fitted into slot 708 (FIG. 5B(ii)) of second scaffold 600.

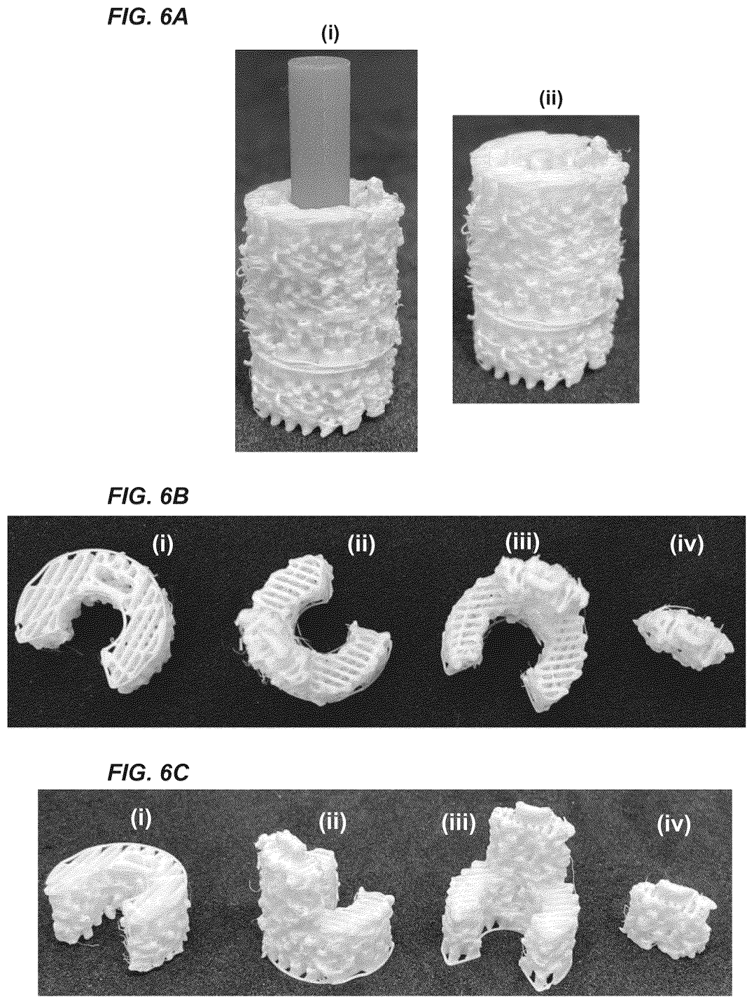

FIGS. 6A-C depict the surgical implant modular assembly described in FIGS. 5A-B. FIG. 6A depicts the surgical implant modular assembly with its parts thereof assembled. FIGS. 6B-C show the top view and perspective view of the individual parts of the surgical implant modular assembly described in FIG. 6A, respectively.

The pin and slot (or hole) described above may advantageously interlock the various segments (first scaffold, second scaffold, alternate scaffold, second alternate scaffold) to achieve structural stability and strength against torsional and translation forces while being implanted within the bone void interface. These forces may be generated during the movement. In an exemplary embodiment, the pin of the first scaffold is fitted into the third slot of the alternate scaffold, when the alternate scaffold is stacked above the second section of the second scaffold.

Without being bound by theory, the surgical implant described herein may further comprise a third scaffold, a fourth scaffold, a fifth scaffold, a sixth scaffold and so forth. The number of scaffolds may be determined based on the dimension (such as length) of the surgical implant required. The number of scaffolds used may not compromise the technical effects and benefits provided by the surgical implant consisting of the first scaffold, second scaffold, alternate scaffold and second alternate scaffold. The third scaffold, fourth scaffold, fifth scaffold, sixth scaffold may structurally be identical to the first and second scaffolds.

For the surgical implant comprising the third scaffold, the third scaffold is also structurally identical to the first scaffold. Additionally, the third scaffold also has a first section and a second section similar to the first scaffold, the first section is positioned above the second section. The second section of the third scaffold has a third opening. When the second scaffold is stacked above the first scaffold and the third scaffold is stacked above the second scaffold, the second opening is configured to complement the first section of the first scaffold such that the first section of the first scaffold fits into the second opening and the third opening is configured to complement the first section of the second scaffold such that the first section of the second scaffold fits into the third opening thereby forming said surgical implant modular assembly. In this configuration, the surgical implant modular assembly may further comprise the alternate scaffold having the third slot such that when the alternate scaffold is stacked above the second section of the third scaffold, the second pin of the second scaffold may be fitted into the third slot of the alternate scaffold.

In some embodiments, the surgical implant modular assembly comprises a bioresorbable material. The bioresorbable material may refer to any materials including metals, alloys, salts, polymers or composites that are biodegradable or bioabsorbable. Specifically, such a material may degrade safely within the body of a subject. In some embodiments, the bioresorbable material may be a porous bioresorbable material. In some embodiments, the porous bioresorbable material may have a pore size of about 0.25 mm to about 4 mm for example 0.25 mm, 0.5 mm, 0.75 mm, 1 mm, 1.25 mm, 1.5 mm, 1.75 mm, 2 mm, 2.25 mm, 2.5 mm, 2.75 mm, 3 mm, 3.25 mm, 3.5 mm, 3.75 mm and 4 mm. The pore size distribution may be homogeneous or heterogeneous throughout or part of the modular assembly of the present disclosure. In some embodiments, the porous structure of the surgical implant may be in the form of filament lines. Advantageously, the porous structure may provide the ability to incorporate autologous bone grafts and biological materials derived from bone marrow aspirates (BMA), platelet rich plasma (PRP) within the porous structure of the surgical implant.

In some embodiments, the bioresorbable material may be a polymer, a salt or a composite. In some embodiments, the bioresorbable material may be a medical grade bioresorbable material. Accordingly, the polymer, the salt or the composite is a medical grade polymer or composite (denoted as “m”). In some embodiments, the polymer may be a polymer comprising polycaprolactone (PCL) or a hydroxyapatite (HA) monomer. In some embodiments, the polymer or the composite comprises a polycaprolactone (PCL)-based polymer. In some embodiments, the salt or the composite may comprise tricalcium phosphate (TCP), particularly β-TCP. In some embodiments, the composite may comprise β-TCP and PCL. In some embodiments, the composite may comprise β-TCP and HA. In some embodiments, the composite may comprise HA and β-TCP that is further mixed with PCL. In some embodiments, the composition of each component in the composite may be adjusted or varied accordingly. In an exemplary embodiment, when the composite is medical-grade PCL and β-TCP, the PCL and β-TCP may be provided in a ratio of 10:90, 20:80, 30:70, 40:60, 50:50, 60:40, 70:30, 80:20 or 90:10 (by weight or volume). In some embodiments, the ratio between PCL and β-TCP is preferably 80:20 (by weight or volume). The ratio may be adjusted to provide suitable mechanical properties and desirable degradation kinetics by hydrolysis. This feature is beneficial as compared to resorption of fast-degrading natural and synthetic polymers.

The inclusion of TCP in the manufacturing of the medical grade of PCL-TCP (mPCL-TCP) composites may increase the osteoconductivity of the scaffolds. This, in turn, may result in the production of a scaffold that provides structural support for cell attachment and tissue development suitable for clinical application in combination with autologous bone grafting. In some embodiments, when the composite comprises TCP and PCL, the composite may further comprise one, two, three or more metals. The metals used may be those found in Groups I and II of the Periodic Table. Preferably, the metals are selected from calcium, magnesium, sodium, potassium and strontium. In some embodiments, the composite is PCL-TCP that further comprises magnesium and this is denoted as PCL-TCP-Mg. In some embodiments, the metal used may be provided in the oxide, peroxide or salt form. In an exemplary embodiment, the PCL-TCP further comprises magnesium sulfate (MgSO₄).

In some embodiments, the surgical implant modular assembly of the present disclosure may be used for treating a bone defect in a subject. Therefore, in some embodiments, there is provided a method for treating a bone defect in a subject.

The present disclosure also provides a surgical implant modular assembly for use in treating a bone defect in a subject, wherein the surgical implant modular assembly comprises:
a first scaffold and a second scaffold, the first scaffold is structurally identical to the second scaffold, each of the first and the second scaffold has a first section and a second section, wherein for each of the first and the second scaffold, the first section is integrated to the second section,
wherein the first section of each scaffold is positioned above the second section of the same scaffold;
the second section of the first scaffold has a first opening;
the second section of the second scaffold has a second opening;
wherein the second opening is configured to complement the first section of the first scaffold such that when the second scaffold is stacked above the first scaffold, the first section of the first scaffold fits into the second opening thereby forming said surgical implant modular assembly.

In some embodiments, the bone defect may be a long bone defect, wherein the long bone is selected from femur, tibia and humerus. In some embodiments, the surgical implant of the present disclosure may facilitate a bony fusion of the critical-sized defect, bone formation inside and outside the fully interconnected scaffold architecture. Additionally, the surgical implant of the present disclosure may provide an osteogenetically inductive and conductive environment paired with mechanical stability (known as diamond concept), which is the key requirement for healing of critical-sized defects. In some embodiments, the surgical implant of the present disclosure may be osteogenetically inductive when incorporated with growth factors or biologics.

In some embodiments, size of each of the first scaffold, the second scaffold, the alternate scaffold and the second alternate scaffold may be advantageously customized or individualized to individual subject. Accordingly, the present disclosure provides a treatment solution for bone defects that is matched to the patient's anatomy to achieve optimal healing due to better fitting and conformity to the unique geometry of bone voids. The customization (including determination of the size) of each of the first scaffold, the second scaffold, the alternate scaffold and the second alternate scaffold may be performed via a Computed tomography (CT) scan. This applies for the third scaffold, fourth scaffold, fifth scaffold, sixth scaffold and so forth. It is to be understood that other suitable scan methods may be used. In some embodiments, the customized scaffolds may be prepared or manufactured using an additive manufacturing for example a 3D printing. This feature may advantageously shorten time-to-surgery by standardizing a universal module, which may be assembled by the surgeons to construct a surgical implant of variable sizes on demand. In some embodiments, a desired length or sizes outside of the individually manufactured length and sizes of the scaffold may be thus manufactured.

In some embodiments, the surgical implant of the present disclosure may be used in conjunction with an intramedullary nail (or rod) as the mechanically most robust implant for long bone stabilization and/or load-sharing with critical-sized defects. Any other suitable fixation techniques than the intramedullary rod may also be used. When the intramedullary nail is used, the customized printing according to a CT-scan may allow for an individualized and optimal fit of the scaffold in the defect and around the nail. Additionally, the 3D-printing in layering technique allows creation of scaffolds having a high porosity with interconnected pores. The porosity of the scaffold may be from about 50% to about 80%, such as 50%, 55%, 60%, 65%, 70%, 75% and 80% (by weight or volume). In some embodiments, the 3D-printing may be using a Fused Deposition Modelling (FDM). In some embodiments, the surgical implant modular assembly of the present disclosure may be used in conjunction with a fixation device including intramedullary nail (or rod) and plates and screws.

In some embodiments, the surgical implant of the present disclosure may be used in long bone reconstruction surgery. In some embodiments, the surgical implant of the present disclosure may be used as bone void filler.

Further, there is provided a method for manufacturing a surgical implant modular assembly, wherein said surgical implant modular assembly comprises:
a first scaffold and a second scaffold, the first scaffold is structurally identical to the second scaffold, each of the first and the second scaffold has a first section and a second section, wherein for each of the first and the second scaffold, the first section is integrated to the second section,
wherein the first section of each scaffold is positioned above the second section of the same scaffold;
the second section of the first scaffold has a first opening;
the second section of the second scaffold has a second opening;
wherein the second opening is configured to complement the first section of the first scaffold such that when the second scaffold is stacked above the first scaffold, the first section of the first scaffold fits into the second opening thereby forming said surgical implant modular assembly;
the method comprises
   providing an image of a bone defect in a subject;
   determining a suitable size of the surgical implant;
   mixing one or more reagents to form each of the first and second scaffold; and
   stacking the second scaffold above the first scaffold.

In some embodiments, following the step of mixing one or more reagent but before forming the first and second scaffolds, there may be intermediary step for forming a composite in the form of pellets. The forming step may comprise milling the composite. The pellets formed may be heterogeneous or homogenous pellets. Following the formation of the composite in the form of pellets, the composite may undergo a melting process by heating the composite followed by extruding the molten composite. The melting may be undertaken via a nozzle and by layer within different axis (including x, y and z axis) of a 3D printer. In some embodiments of the present disclosure, when the surgical implant modular assembly is of cylindrical shape, the method further comprises pressing the surgical implant in circumferential manner on the nail to fill the defect space.

In some embodiments, there is provided a method for manufacturing a surgical implant modular assembly as described herein above, the method comprising:
(i) determining a suitable size of the surgical implant;
(ii) mixing one or more reagents to form each of the first and second scaffold; and
(iii) stacking the second scaffold above the first scaffold.

In some embodiments, prior to step (i), the method further comprises providing an image of a bone defect in a subject. In some embodiments, following the step of mixing one or more reagent but before forming the first and second scaffolds, there may be intermediary step (iia) for forming a composite in the form of pellets. In some embodiments, the forming step in step (ii) may comprise milling the composite. In some embodiments, he pellets formed may be heterogeneous or homogenous pellets. Following the formation of the composite in the form of pellets, the composite may undergo a melting process by heating the composite followed by extruding the molten composite. In some embodiments, the melting may be undertaken via a nozzle and by layer within different axis (including x, y and z axis) of a 3D printer. In some embodiments of the present disclosure, for a surgical implant modular assembly of cylindrical shape, the method further comprises pressing the surgical implant in circumferential manner on the nail to fill the defect space.

In some embodiments, the image of the bone defect may be obtained from any suitable imaging technique including CT scan. In some embodiments, the size (or dimension) of the surgical implant (including the scaffolds) may be determined from the scanning result.

It should be appreciated that the above-described surgical implants and methods of using the same may be varied in many ways, including omitting, or adding elements or steps, changing the order of steps and the type of devices used. It should be appreciated that different features may be combined in different ways. In particular, not all the features shown above in a particular embodiment are necessary in every embodiment of the disclosure. Further combinations of the above features are also considered to be within the scope of some embodiments of the disclosure.

It will be appreciated by a person skilled in the art that the present invention is not limited to what has been particularly shown and described hereinabove. Rather the scope of the present invention is defined by the claims, which follow.

What is claimed is:

1. A surgical implant modular assembly comprising:
a first scaffold and a second scaffold, the first scaffold is structurally identical to the second scaffold, each of the first and the second scaffold has a first section and a second section, wherein for each of the first and the second scaffold, the first section is integrated to the second section,
wherein the first section of each scaffold is positioned above the second section of the same scaffold;
the second section of the first scaffold has a first opening;
the second section of the second scaffold has a second opening;
wherein the second opening is configured to complement the first section of the first scaffold such that when the second scaffold is stacked above the first scaffold, the first section of the first scaffold fits into the second opening thereby forming said surgical implant modular assembly.

2. The surgical implant modular assembly of claim 1, wherein the modular assembly is a cylindrical modular assembly.

3. The surgical implant modular assembly of claim 2, wherein each of the first opening and the second opening is a sideway opening in a range from 60° to 120°.

4. The surgical implant modular assembly of claim 1, wherein the first scaffold further comprises a first pin disposed on top of the first section of the first scaffold and a first slot disposed on bottom of the second section of the first scaffold.

5. The surgical implant modular assembly of claim 1, wherein the second scaffold further comprises a second pin disposed on top of the first section of the second scaffold and a second slot disposed on bottom of the second section of the second scaffold.

6. The surgical implant modular assembly of claim 4, further comprising an alternate scaffold having a third slot such that when the alternate scaffold is stacked above the second section of the second scaffold, the first pin of the first scaffold is fitted into the third slot of the alternate scaffold.

7. The surgical implant modular assembly of any one of claims 5 and 6, further comprising a second alternate scaffold having a third pin such that when the second alternate scaffold is stacked below the second scaffold, the third pin of the second alternate scaffold is fitted into the second slot of the second scaffold.

8. The surgical implant modular assembly of any one of claims 4 to 7, wherein the first pin, the second pin, the third pin, the first slot, the second slot and the third slot, each is of rectangular shape.

9. The surgical implant modular assembly of any one of the preceding claims, wherein the surgical implant modular assembly comprises a bioresorbable material.

10. The surgical implant modular assembly of claim 9, wherein the bioresorbable material is a porous bioresorbable material having a pore size of 0.4 mm to 4 mm.

11. The surgical implant modular assembly of claim 9 or 10, wherein the bioresorbable material is a polymer, a salt or a composite.

12. The surgical implant modular assembly of claim 11, wherein the polymer or the composite comprises a polycaprolactone (PCL)-based polymer.

13. The surgical implant modular assembly of claim 12, wherein the composite is doped with one or more metals.

14. The surgical implant modular assembly of any one of claims 1 to 13, for use in treating a bone defect in a subject.

15. The surgical implant modular assembly of claim 14, wherein the bone defect is a long bone defect selected from the group consisting of femur, tibia and humerus.

16. The surgical implant modular assembly of claim 14, wherein size of each of the first scaffold, the second scaffold, the alternate scaffold and the second alternate scaffold is customized to individual subject.

17. The surgical implant modular assembly of claim 16, wherein the size of each of the first scaffold, the second scaffold, the alternate scaffold and the second alternate scaffold is determined from a Computed tomography (CT) scan.

18. The surgical implant modular assembly of claim 16, wherein the customized scaffold is prepared via an additive manufacturing.

19. A method for treating a bone defect in a subject, comprising inserting the surgical implant modular assembly claimed in claim 1 to the subject.

20. The method of claim 19, wherein the bone defect is a long bone defect selected from the group consisting of femur, tibia and humerus.

21. A method for manufacturing the surgical implant modular assembly claimed in claim 1, the method comprising:
determining a suitable size of the surgical implant;
mixing one or more reagents to form each of the first and second scaffold; and
stacking the second scaffold above the first scaffold, thereby forming the surgical implant modular assembly.
